(19) United States
(12) Patent Application Publication
(10) Pub. No.: US 2010/0291180 A1
(43) Pub. Date: Nov. 18, 2010

(54) NERVE GUIDANCE TUBES

(76) Inventor: Kathryn E. Uhrich, New Brunswick, NJ (US)

Correspondence Address: VIKSNINS HARRIS & PADYS PLLP, P.O. BOX 111098, ST. PAUL, MN, 55111-1098, US

(21) Appl. No.: 12/528,025

(22) PCT Filed: Feb. 20, 2008
(86) PCT No.: PCT/US08/54440

§ 371 Date: Jul. 19, 2010

Related U.S. Application Data
(60) Provisional application No. 60/902,223, filed on Feb. 20, 2007.

(51) Int. Cl. A61F 2/00; A61K 38/18; A61K 31/60; A61P 25/02

(52) U.S. Cl. 424/426; 514/8.3; 514/159

(57) ABSTRACT

Provided herein are cell guidance tubes and methods for their use.

### RELATED APPLICATIONS

This patent document claims the benefit of priority of U.S. Provisional Application Ser. No. 60/902,223, filed Feb. 20, 2007, which application is herein incorporated by reference.

### STATEMENT OF GOVERNMENT SUPPORT

This invention was made with government support under Grant # DE013207 awarded by the National Institutes of Health. The government has certain rights in the invention.

### BACKGROUND

Nerve damage caused by, e.g., traumatic injuries to the limbs, poses an especially challenging medical problem because nerve tissue often fails to heal completely, resulting in permanent disability. There is a need for biomaterials that promote nerve regeneration.

### SUMMARY OF CERTAIN EMBODIMENTS

Certain embodiments of the present invention provide a cell guidance tube that comprises an inner layer, wherein the inner layer comprises at least one biodegradable polymer, wherein the tube comprises a lumen that comprises at least one immobilized peptide mimic of a carbohydrate.

In some embodiments of the invention, the tube further comprises an outer layer, wherein the inner layer and the outer layer each comprise at least one biodegradable polymer.

Certain embodiments of the present invention provide a cell guidance tube that comprises an inner layer and an outer layer, wherein the inner layer and the outer layer each comprise at least one biodegradable polymer.

In some embodiments of the invention, the tube comprises a lumen that comprises a peptide mimic of a carbohydrate. In some embodiments of the invention, the carbohydrate is human natural killer cell epitope (HNK-1) or polysialic acid (PSA).

In some embodiments of the invention, the tube further comprises a middle layer disposed between the inner layer and the outer layer.

In some embodiments of the invention, the middle layer comprises at least one biodegradable polymer.

In some embodiments of the invention, the inner and outer layers have different degradation rates.

In some embodiments of the invention, the inner diameter of the tube is about 2-3 mm.

In some embodiments of the invention, the length of the tube is about 10-30 mm.

In some embodiments of the invention, the tube is formed by extrusion.

In some embodiments of the invention, at least one biodegradable polymer is a bioactive polymer.

In some embodiments of the invention, at least one of the inner, middle, or outer layers comprises a polymer having one or more anti-inflammatory compounds in the polymer backbone. In some embodiments of the invention, the polymer is a polyanhydride. In some embodiments of the invention, the polymer is a polyester or a polyamide.

In some embodiments of the invention, at least one of the inner, middle, or outer layers comprises a salicylic acid-based polymer, a salicylsalicylic acid-based polymer, or a difluorophenyl-salicylic acid-based polymer.

In some embodiments of the invention, at least one biodegradable polymer degrades to release an anti-inflammatory compound.

In some embodiments of the invention, at least one biodegradable polymer degrades to release salicylic acid.

In some embodiments of the invention, at least one biodegradable polymer degrades to release a non-steroidal anti-inflammatory compound.

In some embodiments of the invention, at least one of the inner, middle, or outer layers comprises a polymer having one or more antibiotic compounds in the polymer backbone. In some embodiments of the invention, the polymer is a polyanhydride. In some embodiments of the invention, the polymer is a polyester or a polyamide.

In some embodiments of the invention, at least one biodegradable polymer degrades to release an antibiotic compound.

In some embodiments of the invention, at least one of the inner, middle, or outer layers comprises a polymer having one or more growth factor compounds in the polymer backbone. In some embodiments of the invention, the polymer is a polyanhydride. In some embodiments of the invention, the polymer is a polyester or a polyamide.

In some embodiments of the invention, at least one biodegradable polymer degrades to release a growth factor.

Certain embodiments of the present invention provide a method for regenerate a damaged nerve in a patient (e.g., a mammal, e.g., a human) in need thereof, comprising placing the cell guidance tube of the invention at a site of neuronal injury so as to regenerate the nerve. In some embodiments of the invention, the nerve is a peripheral nerve.

### DETAILED DESCRIPTION

Nerve damage caused by traumatic injuries to the limbs poses an especially challenging medical problem because nerve tissue often fails to heal completely, resulting in permanent disability. Studies have shown that repairing the damaged nerve with synthetic biomaterials can enhance the body's natural capacity for nerve regeneration. However, there is a need for biomaterials that maximize nerve regeneration. As described herein, polymeric materials are investigated for application to nerve repair. These polymers include: biodegradable materials designed to provide temporary structural support while the compromised nerve tissue regenerates, and at the same time reduce inflammation, pain and infection; and collagen gel matrices modified with carbohydrate peptide mimics that can promote functional recovery, e.g., motor or sensory recovery.

Accordingly, certain embodiments provide a cell guidance tube that comprises an inner layer, wherein the inner layer comprises at least one biodegradable polymer, wherein the tube comprises a lumen that comprises at least one immobilized peptide mimic of a growth factor.

Certain embodiments provide a cell guidance tube that comprises an inner layer and an outer layer, wherein the inner layer and the outer layer each comprise at least one biodegradable polymer.

In certain embodiments, the tube further comprises an outer layer, wherein the inner layer and the outer layer each comprise at least one biodegradable polymer.

In certain embodiments, the tube comprises a lumen that comprises a peptide mimic of a growth factor.

In certain embodiments, the growth factor is a nerve growth factor.

In certain embodiments, the growth factor is human natural killer cell epitope (HNK-1) or polysialic acid (PSA).

In certain embodiments, the tube further comprises a middle layer disposed between the inner layer and the outer layer.

In certain embodiments, the middle layer comprises at least one biodegradable polymer.

In certain embodiments, the inner and outer layers have different degradation rates.

In certain embodiments, the inner diameter of the tube is about 2-3 mm.

In certain embodiments, the length of the tube is about 10-30 mm.

In certain embodiments, the tube is formed by extrusion, e.g., melt extrusion.

In certain embodiments, at least one biodegradable polymer is a bioactive polymer.

In certain embodiments, at least one of the inner, middle, or outer layers comprises a polymer having one or more anti-inflammatory compounds in the polymer backbone.

In certain embodiments, the polymer is a polyanhydride.

In certain embodiments, the polymer is a polyester or a polyamide.

In certain embodiments, at least one of the inner, middle, or outer layers comprises a salicylic acid-based polymer, a salicylsalicylic acid-based polymer, or a difluorophenyl-salicylic acid-based polymer.

In certain embodiments, at least one biodegradable polymer degrades to release an anti-inflammatory compound.

In certain embodiments, at least one biodegradable polymer degrades to release salicylic acid.

In certain embodiments, at least one biodegradable polymer degrades to release a non-steroidal anti-inflammatory compound.

In certain embodiments, at least one of the inner, middle, or outer layers comprises a polymer having one or more antibiotic compounds in the polymer backbone.

In certain embodiments, the polymer is a polyanhydride.

In certain embodiments, the polymer is a polyester or a polyamide.

In certain embodiments, at least one biodegradable polymer degrades to release an antibiotic compound.

In certain embodiments, at least one of the inner, middle, or outer layers comprises a polymer having one or more growth factor compounds in the polymer backbone.

In certain embodiments, at least one biodegradable polymer degrades to release a growth factor.

In certain embodiments, the lumen comprises collagen, chitosan, agarose or gelatin.

In certain embodiments, the lumen comprises collagen.

In certain embodiments, the lumen comprises longitudinal channels.

In certain embodiments, at least one of the inner, middle, or outer layers comprises a xylyl-based polymer.

In certain embodiments, at least one of the inner, middle, or outer layers comprises an iodinated salicylate-based polymer.

Certain embodiments provide a cell guidance tube that comprises an inner layer, wherein the inner layer comprises at least one biodegradable polymer, wherein the tube comprises a lumen that comprises at least one immobilized peptide mimic of a carbohydrate.

Certain embodiments provide a method for regenerating a damaged nerve in a patient in need thereof, comprising placing a cell guidance tube as described herein at a site of neuronal injury so as to regenerate the nerve.

In certain embodiments, the nerve is a peripheral nerve.

Certain embodiments provide a tube as described herein for use in medical treatment or diagnosis.

Certain embodiments provide the use of a tube as described herein to prepare a medicament useful for treating a nerve injury in an animal.

An objective of this work is to develop biomaterials to aid in functional recovery from peripheral nerve injury. One objective described herein is to investigate novel biomaterials for the construction of improved bioartificial nerve grafts. The materials investigated herein include two different nerve graft components that have shown promising results in preliminary studies: 1) bioresorbable “PolymerDrugs” that release salicylic acid and/or other nonsteroidal anti-inflammatory drugs (NSAIDs) as they degrade, enabling them to serve not only as a structural scaffolding but also as a drug delivery device, and 2) immobilized peptide mimics of carbohydrates that, e.g., significantly accelerate peripheral motor neuron regeneration in vivo. These materials are evaluated for use in the construction of nerve grafts used to reconnect the proximal and distal segments of the injured nerve. This experimental treatment has been the subject of considerable interest in recent years and is especially appropriate for completely severed nerves. Neuronal and Schwann cell growth in nerve conduits constructed from PolymerDrugs that tested favorably in initial growth and attachment studies are evaluated. The mechanical properties of the nerve conduits are characterized and further optimized to approximate the mechanical characteristics of natural nerve as closely as possible. A collagen gel-based material with covalently coupled peptide mimics, e.g., of two carbohydrates known to accelerate motor neuron regeneration in vivo, human natural killer cell epitope (HNK-1) and polysialic acid (PSA), are evaluated. The gel incorporating the HNK-1 and PSA glycomimetics are used to fill the lumen of the nerve graft. Nerve grafts comprising the PolymerDrug conduit filled with the glycomimetic-modified collagen gel are implanted to evaluate the in vivo regeneration response.

Peripheral nerves consist of parallel bundles of neuronal axons bound together by support tissue into long cables that extend from and innervate regions of the body beyond the brain and spinal cord. Damage to a peripheral nerve cable results in loss of function in the region that it innervates. Loss of sensation and motor control in the limbs can be especially debilitating. Recovery from nerve injury requires repair of the damaged nerve tissue, a challenge that has been partially met by inserting grafts of healthy nerve at the injury site. However, success rates remain less than 80%, with the prognosis dropping off as the severity of the injury increases. In an attempt to improve functional recovery, especially of more serious injuries, researchers have focused on the development of synthetic nerve grafts whose properties can be optimized to promote the healing response.

An important process for functional recovery from nerve injury is the regeneration of damaged axons across the injury site. Schwann cells, the support cells of the peripheral nervous system, aid in this process by clearing debris and producing factors that promote regeneration. A basic function that a nerve graft serves is to provide structural support at the lesion site, acting as a physical bridge across the damaged region for migrating Schwann cells and regenerating axons. In addition, synthetic nerve grafts offer the potential to deliver bioactive components to the wound site, including growth factors, anti-inflammatory drugs, and stem cells. Realizing this potential hinges on the development of new biomaterials with chemical and physical properties that meet the demanding requirements of the nerve repair environment.

A synthetic nerve graft material should: 1) interact favorably with the cellular components of nerve tissue, 2) maintain its integrity during regeneration, and 3) ideally biodegrade when regeneration is complete, leaving the biological structure intact. A number of synthetic materials have shown some preliminary promise for nerve graft construction. However, little systematic evaluation and development of these materials has been undertaken, and none have provided the capability for drug-release. In general, synthetic nerve grafts are formed into tubular conduits and inserted into the nerve at the site of injury. The outer wall of the graft is typically a strong scaffold that can withstand suturing and tension at the site; the best results are obtained when it is filled with an inner scaffold, typically a gel-type material through which cells can grow.

A primary goal of this project is to test new materials for the inner and outer scaffolds of bioresorbable, synthetic nerve grafts. The regeneration response are evaluated in both in vitro and in vivo systems.

This technology is important and could replace donor nerve graft (autograft), such that harvesting a donor nerve is not required. This tube would provide an excellent axon regenerating environment, and we believe the PolyNSAID materials are useful because of their ability to reduce or mitigate inflammation, including neuroma formation. Furthermore, this technology allows for exploring differential cell growth patterns. For example, the exterior conduit surface can be separately modified from the interior conduit surface. Differential degradation rates may induce differential cell types to grow on the interior vs. exterior conduit surfaces. Drug release can be localized differentially to the interior vs. exterior of the conduit.

These guidance tubes could replace the need for autograft that reconnect a nerve gap in peripheral nervous system. Compared to other nerve guidance conduits, the PolyNSAID-based tubes can biodegrade into anti-inflammatory agents that can block the inflammatory cascade and provide a protective, non-scarring environment for nerve repair.

In addition to the ease of handling, some technological advantages provided herein include the ability to: locally control inflammation; control degradation rate; create multi-layer tubes; optionally laser-cut fenestrations on tube ends to assist in suturing.

This project will produce materials with properties useful for synthetic nerve graft construction, including improved strength, flexibility, and/or support for nerve regeneration. The materials may not only promote enhanced cell attachment, survival, and regeneration, but also control pain, inflammation, and infection.

The ability of nerve cells to grown on the PolyNSAID-type materials have been tested. Anti-inflammatory and biodegradable polymers (PolyAspirin® and PolyNSAID®) have been tested for neuron growth. Faster degrading polymers (e.g., based upon salicylic acid and/or salsalate) and slower degrading polymers (e.g., based upon diethylmalonic and/or diflunisal) were tested. Polymers (100 mg/mL) were dissolved in methylene chloride and spin-coated onto glass cover slips (18 mm diameter, 1.5 mm thickness). DRG neuron growth on polymer surfaces was tested for neuron compatibility for up to 3 days. A biodegradable polymer tube has been tested. This polymer tube (1.80 mm inner diameter and 20 mm length) was comprised of a polyanhydride polymer and was assessed by viability of DRG neurons. Notably, cell viability was maintained for up to 5 days. Spin-coated polymers and polymer tubes for neuron growth, and one conduit for nerve growth have been tested. Nerve growth patterns on multiple conduits comprised of polyanhydrides and poly(anhydride-esters) are being tested.

Polymers have been screened for nerve cell morphology and neurite outgrowth. Polymer degradation products (e.g., NSAIDs) do not negatively influence cell growth rates. Tubes have been generated from polymers. Young's modulus (compression) was 200 MPa in dry state and about 200 MPa when hydrated. Nerves are about 0.45 MPa.

Glycopeptide mimics may be conjugated to collagen. In certain embodiments, the mimic may be an 8 amino acid peptide of HNK-1. In certain embodiments, the ubiquitous RGD may be used. In certain embodiments, the bioactivity of the mimic is retained after conjugation.

Suitable compounds and polymers for use in the present invention can be found, e.g., in U.S. Pat. No. 7,122,615, U.S. Pat. No. 6,689,350, U.S. Pat. No. 6,613,807, U.S. Pat. No. 6,486,214, U.S. Pat. No. 6,468,519, U.S. Application Publication Nos. 2005/0089506 and 2003/0104614, and in International Publication Number WO 2004/006863.

Cell morphology and neurite outgrowth length on polymer surfaces can be evaluated to screen for chemistries in 2D context. Cell morphology and neurite outgrowth length on collagen-coated polymer surfaces can be evaluated, as this model would mimic a 3D context. Cell morphology and neurite outgrowth length on glycomimetic collagen-coated polymer surfaces can also be evaluated, as this model would mimic a 3D, biorelevant context.

In certain embodiments of the invention, a bioactive compound may form a portion of the backbone of the polymer backbone. In certain embodiments of the invention, a bioactive compound may be comprised in a matrix formed by the polymer.

In certain embodiments of the invention, the at least 2 of the layers of the tubes have variable degradation rates and/or drug release profiles.

In certain embodiments of the invention, the tubes have about a 1.8 mm inner diameter and are about 20 mm length.

In certain embodiments of the invention, the tubes are extruded from the reaction vessel.

In certain embodiments of the invention, the polymers have unique features: bioresorbable polymers that release nonsteroidal anti-inflammatory drugs (NSAIDs) upon degradation (both structural scaffold and drug delivery system) and immobilized peptide mimics of carbohydrates that accelerate peripheral motor neuron regeneration in vivo (incorporate glycomimetics into lumen of tube).

In certain embodiments of the invention, a PolyNSAID used may be a salicylic acid-based polymer (adipic linker): SAA, e.g.,

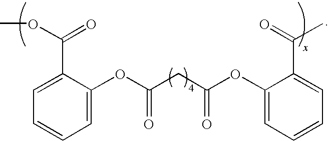

In certain embodiments of the invention, a PolyNSAID used may be a salicylic acid-based polymer (diethylmalonic linker): SAM, e.g.,

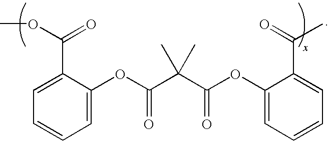

In certain embodiments of the invention, a PolyNSAID used may be a salicylsalicylic acid-based polymer (adipic linker): SA-SA, e.g.,

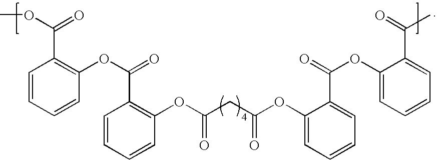

In certain embodiments of the invention, a PolyNSAID used may be a difluorophenyl-salicylic acid-based polymer (adipic linker): DF, e.g.,

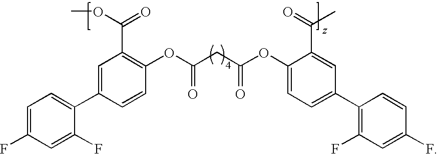

Certain embodiments of the invention provide easily fabricated nerve guidance tubes comprised of biodegradable polymers (e.g., PolyNSAID® and/or PolyAspirin®) to enhance nerve repair. In some embodiments, the biodegradable polymers will release anti-inflammatory drugs upon polymer in vitro degradation.

In certain embodiments, the tube may comprise an inner layer, a middle layer, and an outer layer. The tube can be filled with, e.g., glycopeptide-modified collagen. In certain embodiments, the inner layer is about 200 μm thick. In certain embodiments, the inner layer comprises PLGA (e.g., 80:20) for strength. In certain embodiments, the middle layer is about 100 μm thick. In certain embodiments, the middle layer comprises polyDF for slow release. In certain embodiments, the outer layer is about 100 μm thick. In certain embodiments, the outer layer comprises polySA for fast release. In certain embodiments, the inner lumen is about 0.6 mm, 1.0 mm, 1.5 mm, 2.5 mm, or 3.0 mm. In certain embodiments, the length of the tube is about 3 mm. An example of a tube is provided below. The arrows point to the inner, middle and outer layers.

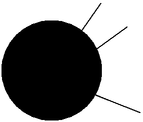

Neuronal and Schwann cell response to bioresorbable biomaterials in a three-dimensional, nerve conduit geometry are evaluated. Regeneration-enhancing peptide mimics, e.g., of the carbohydrates HNK-1 mimic peptide (FLHTRLFV; SEQ ID NO:1), linear scramble peptide control (TVFHFRLL; SEQ ID NO:2) and PSA are evaluated in collagen gels, and the neuronal and Schwann cell response in vitro are evaluated. In vivo testing of tubes filled with glycomimetic-modified collagen gels are evaluated.

Biodegradable Nerve Guidance Conduits Filled with Cell-Inducing Matrix Filler

As described herein, certain embodiments relate to (i) creating tubes from biodegradable polymers (in some embodiments, not bioactive polymers such as our PolyAspirin) (ii) filling the tubes with a matrix that will support cell growth, particularly nerve cells; and (iii) creating longitudinal channels within the matrix to enhance innervation. Filling the polymer tubes can improve mechanical stability and prevent collapse after implantation.

Previous mechanical analyses of conduits made from salicylate-based (also referred to as SAA-based) polymers have shown that the polymer composition can be brittle. As a result, xylyl-based and iodinated salicylate-based polymers were co-extruded with a polyanhydride polymer to fabricate conduits with more favorable mechanical properties. Preliminary mechanical analysis and handling demonstrated that even 10 wt % xylyl-based polymers had mechanical properties useful for a neuronal device. The melt extrusion process currently used to fabricate the conduits allows for a variety of sized hollow tubes. In addition to a protective exterior polymer sheath, an interior biological material channeled matrix will provide the necessary guidance and support for the axons of a regenerating nerve. Potential materials for the interior matrix include: collagen, chitosan, agarose, and gelatin. Initial experimentation involves short conduit segments of 3-5 mm (the length necessary for the rat and monkey model) filled with a channeled collagen matrix. Collagen is one of the main components of the extracellular matrix and has been utilized frequently for nerve regeneration applications. The fibrous nature of collagen allows for permeability and diffusion of molecules. Chitosan is also a biomaterial that has proven to promote adhesion, survival, and neurite outgrowth of nerve cells. Thus, certain embodiments provide processes and products made by the process of melt extruding biodegradable synthetic polymer into a tube and filling the tube with a channeled mechanically supportive biological matrix to promote nerve regeneration.

Current nerve guidance products on the market are hollow tube structures that serve as a protective environment for nerve regeneration but lack the necessary physical structural support for the bands of buenger (Schwann cells). As a result, a channeled matrix composed of a biological material is proposed for the interior of the polymer conduits to maximize the efficiency of axon extension and nerve regeneration.

The biocompatibility of a xylyl-based polymer conduit was compared to two controls: an uncoated glass coverslip and a PLGA (50:50) coated coverslip. PLGA is a well known biocompatible polymer often utilized for biomedical implants. The polymers were dissolved in methylene chloride and spin-coated onto glass coverslips (18 mm diameter). Dorsal root ganglia (DRG) axonal extension was measured for a 7 day period and compared to the original DRG size. The DRGs grew as follows: on the glass surface 827±174%, on the PLGA 776±211%, and on the xylyl-based polymer 641±153%.

The xylyl-polyanhydride copolymer conduit was also mechanically analyzed in compression. A five week study was conducted where samples were incubated in Dulbecco's phosphate buffered saline (DPBS) at 37° C. and 5% CO2 for: 24 hours, 7 days, 14 days, 21 days, 28 days, and 35 days. After 35 days of incubation in DPBS, the Young's modulus decreased from 227±29 MPa to 40±16 MPa. As a comparison, acellular rat sciatic nerve has been reported to have a Young's modulus of ˜580+/−150 kPa.

A broad, overall objective of the work presented herein is to develop advanced biomaterials to aid in functional recovery from peripheral nerve injury. An objective of the project is to investigate novel biomaterials for the construction of improved bioartificial nerve grafts. The materials selected for this study were bioresorbable polymers that have shown promising tissue interactions and bioactivity in other biomedical applications, specifically, new “PolymerDrugs” that release salicylic acid or other nonsteroidal anti-inflammatory drugs (NSAIDs) as they degrade, thus serving not only as a structural scaffolding but also as a drug delivery device. These materials are evaluated for use as nerve conduits, or guidance channels, that can be used to reconnect the proximal and distal segments of an injured nerve. This experimental treatment has been the subject of considerable interest in recent years and is especially appropriate for completely severed nerves. Another possible application would be nerve “patches”, which may be useful for treatment of partially severed nerves, particularly larger diameter nerves.

Four materials were screened for their ability to support the adhesion and growth of neurons and Schwann cells, the two major cellular components involved in nerve regeneration, in a 2D environment (which mimics the nerve patch configuration), and the mechanostructural properties and biocompatiblity were evaluated for nerve conduits formed from these materials. A novel gel matrix was evaluated as a potential interior scaffolding for the nerve conduit.

Nerve damage caused by traumatic injuries to the limbs poses an especially challenging medical problem because nerve tissue often fails to heal completely, resulting in permanent disability. Studies have shown that repairing the damaged nerve with synthetic biomaterials can enhance the body's natural capacity for nerve regeneration. However, there is a need for biomaterials that maximize nerve regeneration. In this project, polymeric materials were screened for potential application to nerve repair. These polymers are biodegradable materials designed to provide temporary structural support while the compromised nerve tissue regenerates, and at the same time promote biological repair by reducing inflammation, pain and/or infection. A new neurite growth-enhancing component of the interior scaffolding of the nerve graft was also examined.

An important process for functional recovery from nerve injury is the regeneration of damaged axons across the injury site. Schwann cells, the support cells of the peripheral nervous system, aid in this process by clearing debris and producing factors that promote regeneration. A basic function that a nerve graft serves is to provide structural support at the lesion site, acting as a physical bridge across the damaged region for migrating Schwann cells and regenerating axons. For this purpose, a synthetic nerve graft material should: 1) interact favorably with the cellular components of nerve tissue, 2) be strong and pliable, and 3) ideally biodegrade when regeneration is complete, leaving the biological structure intact. A number of synthetic materials have shown some preliminary promise for nerve graft construction. However, little systematic evaluation and development of these materials has been undertaken, and none have provided the unique capacity for timed release of anti-inflammatory drugs offered by the materials examined in the current project. These “PolymerDrugs,” represent a class of novel polyanhydrides that hydrolytically degrade into salicylic acid, other nonsteroidal anti-inflammatory drugs (NSAIDs), and/or antibiotics, which can reduce local post-operative inflammation, pain, and infection (e.g., see Erdmann et al., Biomaterials 2000, 20, 1941; Prudencio et al., Macromolecules 2005, 38, 6895; Schmeltzer et al., Polym Bull 2003, 49, 441; Anastasiou et al., J Polym Sci A: Polym Chem 2003, 41, 3667; Schmeltzer et al., Biomacromolecules 2005, 72A, 354). These polymers have been previously evaluated for controlling pain and inflammation associated with periodontal disease and deep bone infections. In addition, Uhrich et al. have determined that the polymers degrade via surface erosion mechanism, an aspect that is attractive for implantable drug delivery systems. The current study focused on the sub-class of salicylic acid-releasing PolymerDrugs.

Recently, molecules that aid in the correct targeting of regenerating peripheral motor axons have been identified by Dr. Schachner. Regrowth of severed peripheral motor axons to improper targets is considered a major reason for poor functional recovery. After lesions of a peripheral nerve, motor axons initially regrow at random into both the proper ‘muscle’ branch and the improper ‘skin’ branch. Two carbohydrates carried by neural cell adhesion molecules, human natural killer cell epitope HNK-1, and 2,8 polysialic acid (PSA), appear to be able to prime regenerating motor axons prior to the branch point and enable the axons to sort into their functionally appropriate tracts. Peptide mimics of these carbohydrates have been developed that similarly enhance correct targeting of the axons and recovery of motor function following a mild (2 mm crush) peripheral nerve injury (Simova et al., Ann Neurol. 2006 October; 60(4):430-7). However the method of application is by injection of the peptides in soluble form, and does not provide for sustained delivery. It is proposed that immobilizing the peptides to a biomaterial scaffold will enhance the effect and enable the results to be extended to larger gaps.

Certain embodiments of the invention will now be illustrated by the following non-limiting Examples.

### Example 1

Neuronal and Schwann cell response was evaluated in vitro for four novel polyanhydride “PolymerDrugs” (Table 1) that hydrolytically degrade into salicylic acid or other NSAID and have been previously shown to control pain and inflammation associated with periodontal disease and deep bone infections. The polymers degrade via a surface erosion mechanism, an attractive feature for implantable drug delivery systems.

Two control surfaces were also evaluated, Poly (D,L-lactide-co-glycolide) (PLGA), a biocompatible polymer, and glass, a standard tissue culture control. Biomaterial surfaces were prepared by dissolving the polymer in methylene chloride at 100 mg/ml, spin-coating onto coverslips (18 mm diam) at 2000 rpm for 30 s, evaporating the methylene chloride in a desiccator for 1 day, then sterilizing for 900 s under UV-light. For neuronal culture, surfaces were incubated with 50 μg/ml laminin for 1 hr prior to plating neurons. Dorsal root ganglia (DRG) explants were dissected from E7-8 chick embryos, placed on the polymer surfaces, and neurite outgrowth was measured at 24 hr. For Schwann cell cultures, rat Schwann cells were placed on the polymer surfaces and cell counts recorded every day for 5 days. The results indicate that neurite outgrowth was similar to the controls for SAA and somewhat reduced for the other three test polymers, though significant in all cases. Schwann cells remained viable on all four test polymers but proliferation was limited, which is desirable for controlling Schwann cell aggregation at the outer scaffold. These results indicate that all four test polymers may be suitable outer scaffolds, with SAA and Diflunisal most favorable for neurite growth.

TABLE 1
BIOMATERIALS EVALUATED

| | | Degradation |
|---|---|---|
| Type | Material | Rate |
| PolyAspirin | SAA | Fast |
| | SAM | Slow |
| PolyNSAID | SA-SA | Fast |
| | Diflunisal | Slow |
| Control | PLGA | — |
| | Glass | — |

### Example 2

### Neuronal and Schwann Cell Response to Novel, Bioresorbable Biomaterials in a Three-Dimensional, Nerve Conduit Geometry

Approach: Neurite outgrowth and Schwann cell migration will be measured as a function of time in tubular nerve conduits formed from the PolymerDrugs described herein, e.g., those listed in Table 1. These will be compared to those for the control polymer, PLGA. In addition, Poly (DTE carbonate), a material with minimal inflammatory potential with ability to support the adhesion and growth of neurons and Schwann cells, will be screened. Mechanical testing will also be performed on the materials.

Plan: Biomaterial tubes (4 mm diam×3 mm long) will be prepared by solvent-casting polymers into an ice-cooled, Teflon-lined tray. Following solvent-evaporation, the flat surfaces will be rolled into tubes and the edges sealed with solvent. The tubes will be filled with a collagen gel matrix to support axonal extension and Schwann cell migration. The collagen gel will be prepared from Vitrogen 100 (Cohesion Co., Temecula, Calif.), a bovine type I collagen that supports nerve regeneration in vitro and in vivo. To prepare the gel, Vitrogen 100 will be neutralized with 0.1 N NaOH and diluted to 2.0 mg/ml collagen using cell culture medium to obtain physiological pH and ionic strength. The collagen solution will be injected into the biomaterial tubes and placed in a 37° C. incubator to permit gelation. In this formulation, the collagen gel represents a non-zero basal control condition that will maintain cell viability while enabling the effects of the biomaterial comprising the outer tube to be evaluated. The collagen gel may be modified to maximally enhance the regeneration response.

To evaluate axonal outgrowth, an E7 chick DRG explant will be sutured in place at the end of the tube and incubated in F12 medium (Gibco, Gaithersburg, Md.) supplemented with D-glucose, L-glutamine, penicillin-streptomycin, insulin, transferrin, selenium, putrescine, progesterone, bovine pituitary extract, and nerve growth factor at 37° C. and 5% CO₂. Axonal growth will be measured at 24-hr intervals over a period of 5 days to determine the growth rate as a function of time. Measurements will be performed by fixing the cylinders in 4% formaldehyde (Fisher Scientific), staining with two primary antibodies, monoclonal anti-neurofilament 200 (Sigma Chemical) and monoclonal anti-neurofilament 160 (Chemicon), then counterstaining with FITC-conjugated goat anti-mouse IgG (green, λ_{exc}/λₑₘ=488/518; Sigma Chemical) to label the neurites. The cylinder will be sliced axially and the length of growth into the tube measured as a function of radial distance from the center of the tube. In separate experiments, primary purified Schwann cells will be placed at the end of the tube by suspending them in a small amount of collagen solution and allowing it to gel on top of one end of the tube. The tubes will be placed in serum-free medium consisting of: 1:1 v/v of DMEM and Ham's F-12 medium (BioWhitaker, Walkersville, Md.) supplemented with 2 mM L-glutamine, 50 U/ml of P/S, 5 μg/ml, insulin, 0.1 mM putrescine, 0.02 progesterone, 0.03 μM sodium selenite, and 8 μg/ml transferrin (all from Sigma Chemical, St. Louis, Mo.). The cultures will be incubated at 37° C. in 5% CO₂. A time course of migration into the tube will be measured by fixing the tubes at 24-hr intervals after seeding, staining the Schwann cell nuclei with ethidium bromide (EtBr), slicing the cylinder axially and measuring the density of nuclei as a function of axial and radial distance in the slice using confocal microscopy. The data for the individual slices will be recombined to obtain axial and radial cell density profiles in the cylinder.

Biomaterial tubes that support neurite outgrowth and Schwann cell survival and migration will be subjected to mechanical testing. The tubes will be tested in quasistatic uniaxial tension with an Enduratec ELF 3200 in its horizontal configuration. All tests will be performed at 37° C. in a humidified Plexiglas chamber. Samples will be clamped at either end with custom-designed compression grips. Alternatively, the tubes can be sutured and glued to the clamps with cyanoacrylate adhesive (Super Glue™). Samples will be stretched to failure at a constant strain rate of 0.5%/sec while continuously recording the force. Elastic modulus, ultimate tensile strength, and strain at failure will be identified from the stress-strain plots. These parameters will be compared to those previously measured for spinal nerve root and acellular sciatic nerve, which indicate that these nerves have a stiffness of ˜1 Mpa. The biomaterials most closely matching the mechanical properties of peripheral nerve will be further optimized by altering synthesis and processing conditions and then re-evaluated for neurite outgrowth and Schwann cell response.

### Example 3

### Immobilization of Regeneration-Enhancing Peptide Mimics in Collagen Gels

Plan: After determining the baseline cellular responses to the conduit biomaterials, the collagen gel in the lumen of the conduit will be modified with growth factors, e.g., regeneration-enhancing peptides. Bovine type I collagen works well as the gel matrix material for several reasons: it is biocompatible (fibroblast-contracted collagen gels are the structural basis for the first living tissue engineered product on the market in the United States (Apligraf, Organogenesis, Inc., Canton, Mass.)), supports nerve regeneration, is easily crosslinked after network formation by a variety of methods that can be used to improve its stiffness, and is amenable to grafting of peptides and molecules to modulate cell behavior. The collagen will be modified by grafting, e.g., covalently grafting, molecules to it that promote neural regeneration.

The effect of two different peptides that mimic neurite growth promoting carbohydrates will be tested: a mimic of polysaialic acid (PSA), and a mimic of the sulfoglucuronyl terminal residue first discovered on human natural killer cells, HNK-1. Polysialic acid has been shown to promote neurite outgrowth and synaptic plasticity; it is expressed mainly during embryonic development and is down regulated in the adult brain except for sites where active neurite outgrowth and neurogenesis occurs. The HNK-1 carbohydrate has been implicated in preferential regrowth and survival of motor axons and motor neurons in peripheral nerve regeneration, respectively. Dr. Schachner has demonstrated promising results on locomotor function with a combination of peptide mimics of these carbohydrates in soluble form via constant infusion following spinal cord injury. Individual application of the carbohydrates did not enhance functional recovery to a significant extent, although a tendency of improved recovery was observed. Peptidomimetics of the carbohydrates, which are more stable and easier to synthesize than the sugars, have proven to be at least as effective as the carbohydrates themselves in in vitro experiments and to be functionally more effective in a dose-response curve than the carbohydrates themselves. The peptidomimetic half-life in vivo is also longer than that of the carbohydrates. The mimics in solution form have been shown individually to enhance peripheral nerve regeneration in a femoral nerve injury mouse model, but regeneration ultimately fails for severe injuries spanning more than several millimeters. It is proposed herein that immobilizing these peptides to a collagen backbone will increase the residence time and availability of the mimics for regenerating axons.

To conjugate the peptide mimics, a 50 mM solution of the peptide will be prepared in distilled water at 50 mM, mixed with 1-ethyl-3-(3-dimethyl aminopropyl) carbodiimide (EDC) at a 5:1 molar ratio, and incubated overnight at room temperature. This will then be added to Vitrogen 100 solution, prepared as described above, with concentrations adjusted to maintain a constant final collagen concentration of 2 mg/ml. The EDC activates the carboxylic group of collagen and forms an amine bond. The adhesion molecule covalently binds to collagen via nucleophilic attack of the carbon of collagen. This technique has been used to successfully graft KRGD peptides (which contain the ubiquitous cell adhesion domain RGD), an RDG scrambled version of this peptide, and bovine serum albumin (BSA) to collagen. A maximum concentration of the grafted peptide is 12.504, and lower concentrations are achieved by mixing grafted, soluble collagen with non-grafted collagen. Higher concentrations can be achieved by altering the initial peptide concentration as warranted. Our experience has shown that RGD peptides at a final concentration of 12.5 μM significantly increase adhesion and adhesion-mediated behavior, such as cell traction, and this will represent the starting point for the peptide mimics.

Neurite and Schwann cell behavior will be evaluated with the gel assay. Schwann cells and sensory neurons from chick DRGs will be acquired. Motor neurons will be isolated from the ventral horns of chick embryo spinal cord according to published protocols. Axon growth and Schwann cell behavior will be evaluated as above for collagen scaffolds with combinations of grafted molecules as described in Table 2. The table includes scrambled versions of the peptide mimics as controls. Each combination will be evaluated at least 4 times. Alternate concentrations/combinations will be assayed as suggested by results from the initial experimental design.

TABLE 2
GLYCOMIMETIC CONCENTRATIONS TO BE EVALUATED

| HNK-1 | Scrambled | PSA | Scrambled |
|---|---|---|---|
| mimic | HNK-1 | mimic | PSA |
| 12.5 μM | | 12.5 μM | |
| 6.25 μM | | 12.5 μM | |
| 12.5 μM | | 6.25 μM | |
| | 12.5 μM | 12.5 μM | |
| 12.5 μM | | | 12.5 μM |
| 12.5 μM | | | |
| 6.25 μM | | | |
| | 12.5 μM | | |
| | | 12.5 μM | |
| | | 6.25 μM | |
| | | | 12.5 μM |

Axon growth will be evaluated through collagen gels with a combination of grafted HNK-1 and PSA mimics. Each row represents a different combination. Ungrafted collagen (no mimics or scrambled mimics) will serve as an additional control.

### Example 4

### In Vivo Testing of Nerve Grafts Constructed Using PolymerDrug Conduits Filled with Glycomimetic-Modified Collagen Gels

Approach: Nerve grafts will be assembled using the best PolymerDrugs identified in the in vitro studies for the outer scaffold. Optimal formulations of the HNK-1, PSA, and HNK-1+PSA peptide mimics identified will be used for the inner scaffold. Nerve grafts will be implanted into adult mice and functional recovery evaluated at 4, 8 and 12 weeks.

Plan: In vivo testing will be performed on adult female C57BL/6J mice (Jackson Laboratory). The left femoral nerve will be exposed and transected 3 mm proximal to the bifurcation of the saphenous and quadriceps branches. The cut ends of the nerve will be inserted into the nerve graft and fixed with single epineural 11-0 nylon stitches, leaving a 2-mm gap between the proximal and distal stump. The skin wound will be closed with 6-0 sutures (Ethicon), and the animals observed for a period of 12 weeks. Three glycomimetic combinations will be tested: HNK-1 mimic only, PSA mimic only, HNK-1+PSA mimics. In addition, three controls, scrambled FINK-1 mimic, scrambled PSA mimic, and unmodified collagen, will be performed, yielding a total of 6 conditions. Nine animals will be tested at each condition. Daily video logs of each animal will be maintained and used to analyze recovery of locomotor function as previously described, by comparing foot angle, limb extension, and step cycle to pre-injury baseline levels. At 4, 8 and 12 weeks following implantation, one-third of the animals will be sacrificed and regeneration will be analyzed at that time point. Femoral nerves will be dissected from animals, fixed by perfusion with formaldehyde and post-fixed in 1% osmium tetroxide in 0.1M sodium cacodylate buffer, pH 7.3, for one hour at room temperature, dehydrated and embedded in resin according to standard protocols. Transverse 1-μm thick sections from the motor and sensory nerve branches will be cut at a distance of approximately 3 mm distal to the bifurcation and stained with 1% toluidine blue/1% borax in distilled water. Total numbers of myelinated axons per nerve cross-section will be estimated on an Olympus microscope equipped with a motorized stage and Microsuite software using a 100× oil objective. Axonal and nerve fiber diameters will be measured in a random sample from each section. A sampling grid with a 60-μm line spacing will be projected onto the microscope visual field. The mean orthogonal diameters of the axon (inside the myelin sheath) and of the nerve fiber (including the myelin sheath) will be measured for all myelinated axons in contact with the vertical grid lines through the sections. The mean orthogonal diameter is calculated as a mean of the line connecting the two most distal points of the profile (longest axis) and the line passing through the middle of the longest axis at a right angle. The degree of myelination will be estimated by the ratio of the axon to fiber diameter. If regeneration is successful for the 2-mm gap, as we anticipate it will be, testing will be repeated for 5- and 8-mm gaps.

### Example 5

Mechanical analyses of conduits made from SAA-based polymers have shown that the polymer composition can be brittle. As a result, xylyl and iodinated based polymers were copolymerized with a polyanhydride polymer to fabricate conduits with more favorable mechanical properties. Preliminary mechanical analysis and handling demonstrated that the 10 w % xylyl based polymers potentially had the mechanical properties that are necessary for such a device. The melt extrusion process currently used to fabricate the conduits allows for a variety of sized hollow tubes. Current nerve guidance products on the market are hollow tube structures that serve as a protective environment for nerve regeneration but lack the necessary physical structural support for the bands of buenger (schwann cells).

As a result, a channeled matrix is proposed for the interior of the polymer conduits. There are several potential materials that could be used for the interior matrix: collagen, chitosan, agarose, and gelatin. Initial experimentation will involve short conduit segments of 3-5 mm (the length necessary for the rat and monkey model) filled with a channeled collagen matrix. Collagen is one of the main components of the extracellular matrix and has been utilized frequently for nerve regeneration applications. The fibrous nature of collagen allows for permeability and diffusion of molecules. Preliminary experiments with 3 mg/ml collagen have shown that the concentration is insufficient in holding its shape after lyophilization. Chitosan is also a biomaterial that has proven to promote adhesion, survival, and neurite outgrowth of nerve cells.

A mold will be made by placing several wires in a sheet of PDMS with tweezers and position hollow tube over the wires (see Scheme 1). The size and amount of wires can be varied. Two wire sizes are currently available: 127 μm stainless steel [Small Parts Inc.] and 203 μm teflon coated wires [Bioabsorbable Therapeutics]. At least ten channels will be created in the scaffolds, while an increasing number of channels may also be utilized. It is expected that there will be a particular threshold where the structural mechanical rigidity of the interior matrix will be forfeited because of too many channels.

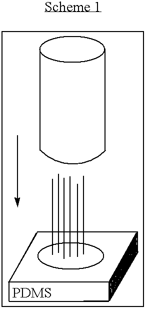

The mold will be filled with the chosen solution and then allowed to gel around the wires. After gelling, the mold will be placed in the −80° C. freezer followed by 12 hours of lypholization at −105° C. and 50 mTorr. The wires will then be extracted from the lypholized material with tweezers and channels will remain in the matrix spanning the entire length of the conduit. The small channels may then be back-filled with another biomaterial through capillary action. The 3 mg/ml concentration will likely be an adequate filling for the channels because previous experiments have shown considerable neuron axon extension on this material. Grafted collagen, e.g., HNK-1 grafted collagen, may be incorporated into the interior matrix of the conduit. Grafting of collagen has thus far been verified with fluorescence and has proven to have a grafting efficiency of ˜50-60%. As a demonstration of using a growth factor, HNK-1 grafted collagen will be an appropriate filling for the channels.

### Example 6

### Bioresorbable Biomaterials and Compatibility with Neurons and Schwann Cells In Vitro

### Materials and Methods

Neurite outgrowth, Schwann cell attachment and Schwann cell proliferation were evaluated in response to four biodegradable polyanhydrides that hydrolytically degrade into salicylic acid: (i) salicylic acid with adipic acid linker (SAA), (ii) salicylic acid with diethylmalonic acid linker (SAM), (iii) salicylsalicylic acid with adipic acid linker (SA-SA) and (iv) diflunisal (DF) with adipic acid linker. Two control surfaces were also evaluated: poly(lactic glycolic acid) (PLGA) a degradable polymer that has been used extensively as an implantable polymer, including for synthetic nerve grafts, and glass, a standard in vitro culture substrate for neurons and Schwann cells. These materials are summarized in Table 3.

TABLE 3
BIOMATERIALS EVALUATED

| Material Type | Polymer |
|---|---|
| Biodegradable | SAA |
| PolymerDrugs | SAM |
| | SA-SA |
| | DF |
| Controls | PLGA |
| | Glass |

Materials and Approach. All solvents, reagents and fine chemicals were purchased from Fisher Scientific (Pittsburgh, Pa.) and SigmaAldrich (Milwaukee, Wis.) unless otherwise indicated. Cells were cultured in a 5% CO₂ incubator (ThermoForma, Steri-Cycle CO₂ incubator, Franklin, Mass.) and eggs were incubated in a circulated air incubator (MODEL 1550, G.Q.F. Manufacturing Co. Savannah, Ga.). Schwann cell and DRG were imaged on an inverted microscope (Olympus IX81, Center Valley, Pa.) and images were captured using a Hamamatsu digital camera system (Olympus IX81, Center Valley, Pa.). Neurite outgrowth and neuron morphology on each surface was observed over 3 days. Schwann cell adhesion, proliferation and cellular morphology on the same polymer surfaces were investigated over 5 days.

Polymer Surface Preparation. Poly (anhydride-esters) (PAE) were synthesized as previously described (Prudencio et al., Macromolecules 2005, 38, 6895; Schmeltzer et al., Biomacromolecules 2005, 72A, 354). These polymers (100 mg) were dissolved in 1 mL of methylene chloride, then spin-coated onto glass coverslips at 2000 rpm for 30 s using a spin-coater (Headway Research, Inc., Garland, Tex.). Before spin-coating, glass coverslips (18 mm diameter; 0.15 mm thickness) were cleaned using Alconox (Alconox Inc., White Plains, N.Y.) and H₂SO₄:H₂O₂ (10:1 v/v) solution and stored in 70% ethanol until use. Coverslips were placed into 12-well plates and sterilized under UV-light at 254 nm for 900 s using a Spectrolinker XL-1500 UV crosslinker (Spectronics Corp., Westbury, N.Y.). Polymer degradation was initiated by adding culture media to each sample and incubating for 1 day at 37° C. PLGA 50:50 copolymer (Boehringer Ingelheim Inc., Germany) was used as a control polymer. Uncoated glass coverslips were also used as a control substrate.

Neuron Isolation. Dorsal root ganglia (DRG) were isolated from E7-8 chick embryos. Specific pathogen-free premium chick eggs were purchased from Charles River Laboratories (North Franklin, Conn.) and maintained in a circulated air incubator. Isolated DRGs explants were placed onto polymer-coated glass coverslips and cultured in neuronal culture medium for 3 days at 37° C. in a 5% CO₂ incubator. Neuronal culture medium consisted of Dulbecco's Modified Eagle's Medium (DMEM)/Ham's F-12 containing 10% v/v fetal bovine serum (FBS; Atlanta Biologicals, Lawrenceville, Ga.), 2 mM L-glutamine, 50 U/mL penicillin/streptomycin (P/S) and 12 ng/mL nerve growth factor (Gibco, Carlsbad, Calif.). To improve neuron attachment, polymer-coated and control coverslips were incubated with 50 μg/mL laminin for 1 hr at 37° C. prior to plating neurons. This is a standard in vitro protocol, since neurons fail to thrive on nearly all surfaces that have not been coated with an appropriate biological attachment factor.

Neuronal Growth. DRG explants on each surface were imaged on an inverted microscope at Day 1, 2, and 3. The extent of neurite outgrowth was analyzed by manually tracing the neurite outgrowth halo around the explant and calculating its mean diameter using Microsuite™ imaging software (Olympus, Soft Imaging Program, Center Valley, Pa.). Neurite outgrowth length was calculated as ½×(mean diameter of the outgrowth halo minus mean diameter of the explant body). On Day 1, outgrowth morphology was also assessed using a score-based morphological measure. Explants that successfully attached to the surface and showed neurite outgrowth were assigned a score of 3; explants attached with slight outgrowth were scored 2; explants that attached to the surface but showed no outgrowth were scored 1; explants that detached from the surface were scored 0.

Immunostaining was used to improve the visualization of neurons on polymer-coated surfaces. Cultures were fixed in 4% paraformaldehyde solution for 20 min at 25° C. then extensively washed in immunobuffer consisting of 1% bovine serum albumin and 0.5% Triton X-100 in phosphate buffered solution (MP Biomedicals, Inc., Solon, Ohio). The samples were blocked with 10% goat serum in immunobuffer for 1 hr. Samples were incubated in primary antibody solution, a mixture of neurofilament 68 (1:5000) and 200 (1:1000), for 1 hr at 25° C., followed by secondary antibody incubation with anti-mouse Alexafluor 546 (1:400) (Molecular Probes, Carsbad, Calif.) for 45 min at 25° C.

Schwann Cell Preparation. Purified primary rat Schwann cells were cultured for 5 days on the PAE-coated, PLGA-coated and uncoated glass coverslips. The Schwann cells were maintained in DMEM media with 10% FBS, glutamine and P/S. Prior to plating, cells were detached by incubating in trypsin (0.02 mg/mL) at 37° C. for 5 min, and plated onto prepared surfaces at 1×10⁵ cells/well.

Schwann cell attachment and proliferation. Schwann cell numbers for each sample surface were evaluated by direct cell count. Nine specific locations of evenly spaced intervals for each sample were selected using the Microsuite™ program. Over a 5 day time period, cells in the nine locations on each sample were imaged daily at 10× magnification on the inverted microscope (Olympus IX 81). All experiments were repeated five times.

Statistical Data Analysis. Ten experimental data sets for neurite outgrowth on the polymer surfaces and five sets for Schwann cell proliferation were subjected to statistical analysis using Excel (Version 2003; Microsoft, Redmond, Wash.) and Statanalysis (www.DanielSoper.com). Student t-test and one-way analysis of variance (ANOVA) analysis were performed using a 95% confidence limit (p<0.05).

### Results and Discussion

Qualitative DRG Morphology on Polymer Surfaces. Explants on SAA and DF surfaces exhibited morphological characteristics similar to healthy explants seen on PLGA and glass coverslip controls, with significant neurite outgrowth extending from the explant perimenter. DRG's on SAM and SA-SA surfaces displayed much shorter neurites.

Initial DRG Response. The best response was seen for neurons on the SAA test polymer and on glass and PLGA controls, indicating attachment and extensive growth. Although the mean score for neurons on SAA was slightly lower than on the controls, the difference was not significant. Significantly lower scores were observed on SAM, SA-SA and DF surfaces.

DRG Neuron Growth on Polymer Surfaces. Further neuron response analysis was performed by measuring neurite outgrowth length over three days on each surface. In general, neurite length increased daily over the time period for all surfaces. Neuron growth on the four NSAID-based polymers was compared to neuron growth on a PLGA surface. Neurons on SAA surfaces showed the highest outgrowth and remained consistent with the outgrowth on the PLGA control throughout the culture time. The average outgrowth rates on SAA and 50:50 PLGA control surfaces were 583 and 553 μm/day, respectively. Neurite length on DF-based polymer surfaces was significantly lower on Days 1 and 2 compared to the PLGA control, with no significant difference by Day 3. Neurons on SAM and SA-SA generally showed daily growth at all time points; neurite outgrowth ranges were significantly different from PLGA and uncoated glass controls. These results suggest that SAA is a neuron-compatible polymer and a potential candidate for a nerve guidance material. DF demonstrates less compatibility than SAA, but still may be sufficient with further modification (e.g., coating with adhesive proteins).

Schwann Cell Attachment and Proliferation. The degree of Schwann cell attachment on each of the surfaces at Day 1 was evaluated. The only significant difference observed among the different surfaces was a reduced attachment to SA-SA. Cell proliferation was followed for five days. Cells on the control surfaces demonstrated a typical proliferation pattern, with cells rapidly proliferating up to the first 3 days followed by a slower growth rate. Cell numbers were significantly lower on PLGA than on the uncoated glass control. Cell numbers on SAA and DF were nearly constant with time. Cell numbers were lowest on SA-SA and SAM surfaces, even showing a slight decrease over the culture time period. All polymers in this study, including PLGA, degrade upon contact with the media; it is possible that the degradation by-product inhibits cell proliferation to varying degrees, depending on the polymer.

Schwann Cell Morphology. Schwann cell morphology on the controls (50:50 PLGA and uncoated glass coverslip) showed typical Schwann cell morphology: spindle-shaped (bi-polar or tri-polar) and well flattened with oval nuclei. On SAA and DF, most cells exhibited spindle-shaped morphology, similar in appearance to the controls; however, cells tended to be more extended and lower cell numbers were observed. SAM surfaces had mixed regions of cell spreading, some cells similar in appearance to controls, and some more rounded cells that indicated lower surface compatibility. Schwann cells on SAM surfaces typically displayed a more rounded shape, and cells tended to form clusters. Cells on the SA-SA surfaces were primarily rounded.

### Example 7

### The Structure, Mechanical Strength and Biocompatibility of Nerve Conduits

Of the different test polymers evaluated, the best cellular response was clearly obtained with the salicylic acid-based poly (anhydride-ester) with adipic linker (SAA). Thus, SAA was selected for further testing. Tubular conduits of SAA-based polymers were successfully fabricated with a melt extrusion method. Scanning electron microscopy was used to observe their structural integrity and compression testing was performed to quantify their mechanical properties. Although biocompatibility with neurons and Schwann cells at early times was demonstrated in Example 6, the times examined preceded the release of significant quantities of salicylic acid from the polymer. Therefore, in this aim, we also investigated the effects of released salicylic acid on Schwann cells and neurons.

### Materials and Methods

Materials. All media components, cell culture and immunostaining reagents were purchased at Sigma (St. Louis, Mo.), and cell flasks and well plates were purchased from Fisher Scientific (Fair Lawn, N.J.), unless otherwise noted. Nerve guidance tubes were manufactured at Bioabsorbable Therapeutics (Menlo Park, Calif.) using a melt extrusion process.

Scanning Electron Microscopy of Conduits. The conduit structure was visualized using scanning electron microscopy (SEM; AMRAY 1830 I, Bedford, Mass.) and fluorescence microscopy (Olympus IX 81, Center Valley, Pa.). Conduits were cut into 3 mm lengths using a hot Accu-Knife™ (Control CO, Houston, Tex.) warmed with a heating gun. For fluorescence microscopy, conduits were observed at 350, 490 and 557 nm. For SEM observation, tubes were affixed to specimen mounts (Electron Microscopy Sciences, Fort Washington, Pa.) using nonconducting adhesive tabs (Electron Microscopy Sciences). Cross-section and surfaces of samples were gold-coated using a Sputter Coater (BALZER SCD 004; Baltec, Tuscon, Ariz.) and examined at an electron voltage of 20 kV.

Compression Testing. Mechanical analysis of polymer conduits was performed with a Perkin-Elmer DMA 7e dynamic mechanical analyzer with a TAC 7/DX thermal analysis controller (Waltham, Mass.). PE Pyris software (Version 3.81, Perkin Elmer, Waltham, Mass.) associated with the DMA was used for collecting and processing data. Conduit samples (2.7 mm×1.8 mm i.d., n=3) were loaded on the DMA and a perpendicular force applied to the longitudinal axis of the tubes at a 25 mN initial load ramped to 8000 mN at a rate of 200 mN/min. Temperature was maintained at 22° C. and the data exported to Excel for further analysis. Young's modulus was calculated as the slope of the initial linear portion of the stress-strain curve.

Analysis of In vitro Degraded Media. To evaluate the influence of released drug on DRG neurons and Schwann cells, the polymer conduits were hydrolytically degraded in medium composed of Dulbecco's Modified Eagle's Medium (DMEM), 10% v/v fetal bovine serum (FBS, Atlanta Biologicals, Lawrenceville, Ga.), 2 mM L-glutamine, and 50 U/mL penicillin/streptomycin. Three different conduits (2 cm length) were placed onto individual centrifuge tube and incubated with 3 mL of media at 37° C. Media was collected on Day 1 (10% release), Day 4 (50% release), and Day 7 (100% release). Medium without released drugs was used as a control.

DRG Neuron Isolation and Culture in Polymer Degradation Media. DRG neurons were removed from E7-8 chick embryos (Charles River Laboratories, North Franklin, Conn.) and placed in cold Hank's Buffered Salt Solution (BioWhittaker™, Combrex BioScience, Walkerville, Md.). Ganglia were dissociated by incubation in 0.25% trypsin for 10 min at 37° C. and triturated ten times with a fire-polished pipette.

Laminin (100 μL/well) was added to 24-well plates and incubated for 1 hr at 37° C. to coat the plate surfaces. Dissociated neurons were seeded at 5×10⁴ cells/well. Nerve growth factor (R&D Systems, Minneapolis, Minn.) was added at 12 ng/mL. Media were collected (10, 50 and 100% drug released media) and added at a volume of 500 μL per well and neurons were cultured for 24 hr in an incubator (37° C. and 5% CO₂; ThermoForma, Franklin, Mass.). All experiments were performed in triplicate.

Immunostaining and Image Analysis. After 24 hr, neurons were immunostained against neurofilament antibody. Cells were fixed with 4% paraformaldehyde for 15 min at 25° C. and extensively washed in an Immunobuffer solution. Immunobuffer solution consisted of 1% bovine serum albumin and 0.5% Triton X-100. Non-specific binding was blocked by 1 hour incubation with 10% goat serum in immunobuffer. The primary antibody was prepared as the mixture of monoclonal anti-neurofilament 68 (1:5000) and 200 (1:1000) and incubated for 1 hour at 25° C. Secondary antibody, anti-mouse Alexafluor 546 (1:400; Molecular Probe, Carsbad, Calif.), was incubated for 45 min at 25° C. After three washes with Immunobuffer, immunostained neurons were imaged with fluorescent microscopy in red fluorescent filter range (546 nm). To investigate neuron growth, morphology and neurite outgrowth were observed in images. Neurite outgrowth length was measured from the cell body to the neurite tip. Neurite lengths were measured and averaged (n=3) using Microsuite™ (Olympus, Soft Imaging Program, Center Valley, Pa.).

Schwann Cell Isolation and Culture. Primarily isolated Schwann cells were maintained in medium composed of DMEM, 10% FBS, 2 mM glutamine, and 50 units of penicillin and streptomycin. Confluent cells were detached from the flask by trypsin (0.25 mg/mL) incubation for 5 min at 37° C., and placed in media at 5×10⁴ cells/well. Schwann cells in degradation media (10, 50 and 100% degradation) and control medium were maintained for 3 days. Schwann cell morphology was observed at Days 1, 2, and 3 with light-microscopy (Olympus IX 81, Center Valley, Pa.). To compare cell numbers between samples, multiple images were taken per sample and cell numbers were counted in each image using Microsuite™. All experiments were performed in triplicate.

Statistical Analysis. Neurite outgrowth and Schwann cell proliferation in degradation media were compared to cells in the control medium using a one-way analysis of variance (ANOVA; Statanalysis, www.Daniersoper.com) Significance was determined at p<0.05.

Results and Discussion

Nerve Guidance Conduit Structure. Representative SEM images of the polymer conduits were prepared. Images at high magnification show a relatively smooth cross-section, and the inner and outer surfaces also appear generally smooth. As surface roughness has been shown to affect peripheral nerve regeneration and smoother surfaces demonstrated improved regeneration, these conduits are promising. Nerve guidance conduits were autofluorescent at 350, 490 and 557 nm the most frequently used wavelengths for biological work.

Nerve Guidance Conduit Mechanical Strength. Preferably, nerve conduits should maintain a fixed shape with sufficient mechanical strength to support neuron growth, glial cell attachment and proliferation. In addition, nerve conduit should maintain a stable path across the injury site to protect neuron growth, which again depends on the mechanical integrity of the conduit. Because the nerve grafts are bridged without obvious tension into the nerve gap, compression tests are accepted to evaluate mechanical strength (Belkas et al., Biomaterials. 26:1741-1749, 2005). Compression tests on the polymer conduits yielded a Young's modulus of ˜200 MPa for both dry and wet form of the conduit. For comparison, a Young's modulus of tibial nerve is ˜0.4 MPa. For other synthetic materials, Young's modulus values are ˜660 MPa for poly(L-lactide), ˜0.28 MPa for poly(glycerol sebacate) and ˜260 MPa for poly(hydroxyl butylate). Although the mechanical strength of the polymer conduit is higher than natural nerve, it is closer in value than other synthetic materials. Cytocompatibility of Conduits with DRG Neurons. To evaluate the influence of the polymer degradation products on neuron growth, conduits were incubated in the media for 1, 4, and 7 days aiming for approximately 10, 50 and 100% drug release based upon previously published data (Schmeltzer et al., Biomacromolecules 2005, 72A, 354). SAA degrades into salicylic acid and adipic acid with incubation. Drug amounts were calculated at 0.2, 1.1 and 2.2 mg/mL in 10, 50 and 100% media, respectively, based on conduit weight. Neurons were cultured in the degradation media for 24 hr and media alone was used as the negative control. The incubation time was chosen because neurons attach to the surfaces and initiate neurite outgrowth within 24 hours. No significant differences were observed for neurite lengths in degradation media and control media.

Neuron Morphology in Polymer Degradation Media. Neuron morphology in degradation media was also observed after 24 hr. Representative images of dissociated neurons in 50% and 100% polymer degradation media and in control media were prepared. Neurons were immunostained with anti-neurofilament so that neuron bodies and neurites appear as bright circles and thin lines, respectively. The immunostained images show typical neuron morphology; neurons spread and uniformly distribute on the surfaces with extended neurites. No obvious difference between the samples and the control were observed in neuron morphology, which reinforces the cytocompatibility of polymer degradation products.

Schwann Cell Proliferation in Polymer Degradation Media. Schwann cells support nerve regeneration by myelinating neurons and secreting neurotrophic factors. Thus, Schwann cell response to the degradation media is an important test for potential nerve conduits. Schwann cells were cultured for 3 days in the polymer degradation media and control media. The three days of culture time is sufficient to observe cell adhesion and proliferation. Over the three days, cells revealed typical cell growth curve at all samples. The average cell numbers increased two-fold from Day 1 to Day 3, indicating at least one growth cycle. For example, cells in 100% polymer degradation media increased from 50% on Day 1 to 100% on Day 3. No significant difference (p<0.05) among 10, 50, and 100% degradation media and control media was observed.

Schwann Cell Morphology in Polymer Degradation Media. Schwann cell morphology in polymer degradation media was further observed. No notable differences in cell morphology were observed among the samples and controls. In both the degradation and control media, cells homogenously displayed typical bi-polar or tri-polar stellate morphology of Schwann cells with oval nuclei and lamellipodial extensions. Cell clustering, cell lysis, detachment from the well plates, or abnormal cellular morphology was not observed. Biocompatibility of the polymer degradation products to Schwann cells was demonstrated by the normal proliferation rate and normal morphology. Thus, the conduits are expected to positively impact Schwann cell activities.

### Example 8

### A Novel Gel Matrix as an Interior Scaffolding

Materials and Methods.

Collagen Scaffold Preparation. Collagen scaffolds were prepared by neutralizing a 3 mg/ml solution of type I collagen (Vitrogen, Cohesion Technologies, Temecula, Calif.) dissolved in 0.2N acetic acid with 0.1N NaOH (Enever et al., J Surg Res, 2002. 105(2): p. 160-72). Collagen gels were diluted to 2 mg/ml collagen using 10×MEM and M199 supplemented with L-glutamine and penicillin/streptomycin to obtain physiological pH and ionic strength. The collagen solution was placed in a 37° C. incubator to permit self-assembly.

Adhesion Molecule-Grafted Collagen Preparation. HNK-1 peptide mimic-grafted collagen were prepared by replacing M199 and L-glutamine in the above recipe with the peptide of interest. Briefly, a protein/1-ethyl-3-(3-dimethyl aminopropyl) carbodiimide (EDC) solution was prepared at a 1:1 molar ratio and incubated overnight at room temperature. The EDC activates the carboxylic group of the protein and forms an amine bond. This solution was added to the normal collagen recipe described above to prepare grafted collagen, with concentrations adjusted to maintain a constant final collagen concentration of 2 mg/ml. The peptide-grafted collagen self assembled into a gel upon incubation at 37° C. FITC-conjugated FINK-1 was used to verify grafting using fluorescence microscopy. Following self-assembly, the gels were rinsed for 8 days to remove any unbound fluorsescence label.

Cell Culture. Ventral spinal cord explants from Day 8 chick embryos were cultured in HNK-1 mimic-grafted gels and control collagen gels for 4 days. Some explants were also cultured in FINK-1 mimic gradients formed using a microfluidics system.

### Results and Discussion

HNK-1 Grafted Collagen Gels. The covalent coupling of HNK-1 peptide mimic to soluble collagen successfully immobilized the FINK-1 mimic to the fibrillar collagen matrix following collagen gel formation. Use of FITC conjugated mimic and extensive rinsing of the collagen gel following self assembly ensured that any fluorescence was due to HNK-1 mimic incorporated into the matrix and not to unbound mimic.

Neurite Extension. Data indicates that including the HNK-1 peptide within a Type I collagen gel enhances outgrowth of axons from chick embryo spinal cord explants. Neurite extension was observed for both HNK-1 mimic grafted gels and collagen control gels, but was greater for FINK-1 mimic gels. These results demonstrate that HNK-1 mimic grafted gels support neurite growth, and indicate that axon extension may be improved in biomaterials with immobilized HNK-1/HNK-1 mimics. In another experiment, when presented with a gradient of this grafted peptide via a microfluidics assay, axons from spinal cord explants preferentially grew up the gradient of HNK-1 mimic.

### Example 9

### Synthetic Biodegradable Polymer Tubes

To overcome the potential issue of NSAID-intolerance, alternate polymers that do not biodegrade into therapeutics were explored. For this specific aim, xylyl-based polymers were used (Anastasiou et al., Macromolecules 33:6217-6221 (2000)). The xylyl based polymers were compared to PLGA (50:50) as well to the SAA-based polymer.

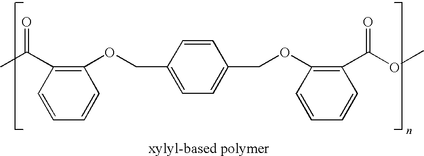

### Materials and Methods

Substrate Preparation. Glass coverslips [18 mm] (Fisher, Phillipsburg, N.J.) were prepared by spin-coating. Polymer (50 mg) was dissolved in 1 mL of methylene chloride. Polymer solution (80 μl) was added to each glass coverslip and spun at 200 rpm for 40 seconds using a humidified spin-coater (Headway Research, Inc., Garland, Tex.). Prepared glass coverslips were placed in a 12-well plate and collagen gel solution was added. Collagen gel was prepared by mixing 20 μl of M199, 1 μl of penicillin/streptomycin, 10 μA of L-glutamine, and 677 μl of 3.0 g/mL Vitrogen collagen solution (Cohesion Corp., Temecula, Calif.). The collagen network self-assembled into a gel when incubated at 37° C. and 5% CO₂. The experimental design of a mimetic NGC environment is shown in Scheme 2, which shows a cross-section of a tube with appropriate dimensions projected into an experimental model.

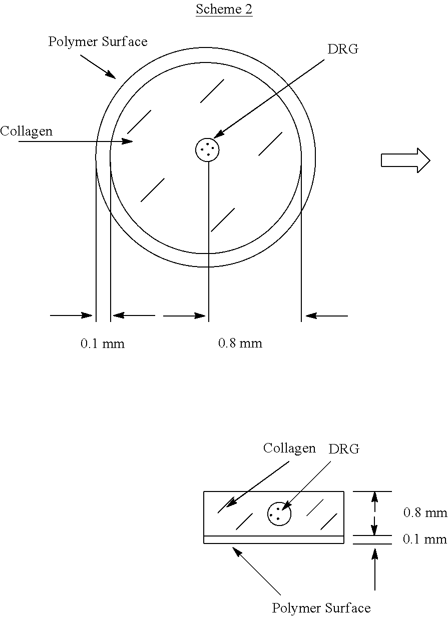

Cell Culture. Dorsal root ganglia (DRG) were extracted from embryonic day 8 (E8) White Leghorn chick embryos (Charles River, Wilmington, Mass.) and seeded into the three-dimensional collagen gel. DRG media consists of Dulbecco's Modified Eagle's Medium (DMEM; Sigma, St. Louis, Mo.), 10% v/v fetal bovine serum (Atlanta Biologicals, Lawrenceville, Ga.), penicillin/streptomycin, L-glutamine, and nerve growth factor.

Neurite outgrowth was observed on day 1, 4 and 7 with a fluorescent microscope (Olympus IX81, Center Valley, Pa.). The circumscribed ‘body’ region is compared to the exterior growth area using Microsuite™ software. After 7 days of growth, the final axonal measurement was obtained by fixing the DRG with 4% formaldehyde (Fisher Scientific), staining with monoclonal anti-neurofilament 200 [1:200] (Sigma) and monoclonal anti-neurofilament 68 [1:1000] (Sigma) and counterstaining with anti-mouse Alexa Fluor 546 [1:100](Molecular Probes, Carlsbad, Calif.) for labeling the neurite extensions.

Mechanical Testing. Mechanical analysis of the polymer conduits was performed with a Bose EnduraTEC ELF 3200 (Framingham, Mass.). Compression testing was performed on short cylinder segments using 5 cm diameter parallel plates and a 50 lb load cell. All testing was performed at room temperature at a crosshead speed of 1.0 mm/min. The polymer conduits were compressed in the radial direction and had a length ˜3 mm. Data acquisition (applied force and displacement) was performed with the WinTest software. The strain (c) of the conduit is defined as:

$$ɛ = \frac{{d}_{o} - d}{{d}_{o}} ,$$

where dₒ is the initial outside diameter of the tube and d is the diameter after compression.

Young's modulus values (E) were calculated using the following equation:

$$F = \frac{32 π EI}{( {π}^{2} - 8 ) {d}_{o}^{2}} ( ɛ ) ,$$

where I is the second moment of area defined as:

$$I = \frac{π ( {r}_{o}^{4} - {r}_{i}^{4} )}{4} .$$

### Results and Discussion

Cytocompatibility. DRGs in the NCG environment successfully adhered and showed outgrowth. When initially seeded into the collagen gel, the DRG is simply a round body with no axonal outgrowth. Polymer biocompatibility is indicated by the extent of axonal projections (the halo surrounding the dense core). A larger halo of axons typically represents a more viable environment for neuron growth. There is a general trend of an increasing axonal halo as time progresses.

Results show an overall increase of neurite outgrowth size compared to the original DRG body size for the xylyl-based polymer compared to PLGA and the uncoated glass control.

Mechanical Properties. A five week study was conducted where samples were incubated in Dulbecco's phosphate buffered saline (DPBS) at 37° C. and 5% CO2 for: 24 hours, 7 days, 14 days, 21 days, 28 days, and 35 days.

### SUMMARY

The biocompatibility of salicylic acid-based polymers was evaluated by culturing DRG neurons and Schwann cells on the polymer surfaces. Evaluated in terms of neuronal growth, Schwann cell adhesion and proliferation, the SAA polymer was the most promising, followed by DF. SAA demonstrated neuronal growth similar to the PLGA control, while both SAA and DF exhibited Schwann cell adhesion and proliferation rates similar to PLGA.

Tubes formed from SAA exhibit mechanostructural properties suitable for use as nerve conduits. Degradation products released from these tubes had no adverse effect on neuronal growth, or Schwann cell adhesion and proliferation.

HNK-1 peptide mimic grafted in a 3D collagen gel matrix enhanced neurite outgrowth. The degree of grafting and the response of isolated mouse spinal cord and dorsal root ganglion neurons through gels grafted with either HNK-1 or PSA mimics will be evaluated, the dual presentation of the mimics in vitro will be optimizing, and then testing the optimum biomaterial in vivo in a mouse peripheral nerve injury model. The degradable xylyl-based polymer tubes will be filled with a mechanically supportive matrix filler (eg, collagen or chitosan) and channels appropriate for innervation will be created using 100 micron-sized rods.

### LITERATURE

1. Capturing the Full Power of Biomaterials for Military Medical Needs. National Academies Press, Washington, D.C., 2004.
2. Schmidt C E, Leach J B. Neural tissue engineering: strategies for repair and regeneration. Annu Rev Biomed Eng. 2003; 5:293-347.
3. Erdmann, L; Uhrich, K. Synthesis and degradation characteristics of salicylic acid-derived poly(anhydride-esters). Biomaterials 2000, 20, 1941.
4. Schmeltzer, R; Uhrich, K. Synthesis and Characterization of Salicylic Acid-based Poly(anhydride-ester) Copolymers. J Bioactive Compat Polym accepted.
5. Prudencio, A; Schmeltzer, R; Uhrich, K. Effect of linker structure on salicylic acid-derived poly(anhydride-esters). Macromolecules 2005, 38, 6895.
6. Schmeltzer, R; Anastasiou, T; Uhrich, K. Optimized synthesis of salicylate-based poly(anhydride-esters). Polym Bull 2003, 49, 441.
7. Anastasiou, T; Uhrich, K. Aminosalicylate-based biodegradable polymers: Synthesis and in vitro characterization of poly(anhydride-esters) and poly(anhydride-amides). J Polym Sci A: Polym Chem 2003, 41, 3667.
8. Schmeltzer, R; Schmalenberg, K; Uhrich, K. Synthesis and cytotoxicity of salicylate-based poly(anhydride-esters). Biomacromolecules 2005, 72A, 354.
9. Schmeltzer, R; Uhrich, K. Synthesis and Hydrolytic Degradation of Antiseptic-based Poly(anhydride-esters). Biomacromolecules under review.
10. Erdmann, L; Macedo, B; Uhrich, K. Degradable poly(anhydride-ester) implants: Effects of localized salicylic acid release on bone. Biomaterials 2000, 21, 2507.
11. Harten, R; Svach, D; Schmeltzer, R; Uhrich, K. Salicylic acid-derived poly(anhydride-esters) inhibit bone formation and resorption in vivo. J Biomed Mater Res: Part A 2005, 42A, 354.
12. Whitaker-Brothers, K; Uhrich, K. Poly(anhydride-ester) fibers: Role of copolymer composition on hydrolytic degradation and mechanical properties. J Biomed Mater Res 2004, 70A, 309.
13. Whitaker-Brothers, K; Uhrich, K. Investigation into the Erosion Mechanism of Salicylate-based Poly(anhydride-esters). J Biomed Mater Res: Part A in press.
14. Tsai E C, Dalton P D, Shoichet M S, Tator C H. Synthetic hydrogel guidance channels facilitate regeneration of adult rat brainstem motor axons after complete spinal cord transection. J. Neurotrauma. 2004 June; 21(6):789-804.
15. Evans G R, Brandt K, Katz S, Chauvin P, Otto L, Bogle M, Wang B, Meszlenyi R K, Lu L, Mikos A G, Patrick C W Jr. Bioactive poly(L-lactic acid) conduits seeded with Schwann cells for peripheral nerve regeneration. Biomaterials. 2002 February; 23(3):841-8.
16. Evans G R, Brandt K, Niederbichler A D, Chauvin P, Herrman S, Bogle M, Otta L, Wang B, Patrick C W Jr. Clinical long-term in vivo evaluation of poly(L-lactic acid) porous conduits for peripheral nerve regeneration. J Biomater Sci Polym Ed. 2000; 11(8):869-78.
17. Tai H C, Buettner H M. Neurite outgrowth and growth cone morphology on micropatterned surfaces. Biotechnol Prog. 1998 May-June; 14(3):364-70.
18. Thompson D M, Buettner H M. Oriented Schwann cell monolayers for directed neurite outgrowth. Ann Biomed Eng. 2004 August; 32(8):1120-30.
19. Thompson D M, Buettner H M. Schwann cell response to micropatterned laminin surfaces. Tissue Eng. 2001 June; 7(3):247-65.
20. Dubey, N., P. C. Letourneau, and R. T. Tranquillo, Guided neurite elongation and schwann cell invasion into magnetically aligned collagen in simulated peripheral nerve regeneration. Exp Neurol, 1999. 158(2): p. 338-50.
21. Ceballos, D., X. Navarro, N. Dubey, G. Wendelschafer-Crabb, W. R. Kennedy, and R. T. Tranquillo, Magnetically aligned collagen gel filling a collagen nerve guide improves peripheral nerve regeneration. Exp Neurol, 1999. 158(2): p. 290-300.
22. Singh A, Lu Y, Chen C, Cavanaugh J M. Mechanical properties of spinal nerve roots subjected to tension at different strain rates. J Biomech. 2006; 39(9):1669-76. Epub 2005 Jul. 5.
23. Borschel G H, Kia K F, Kuzon W M Jr, Dennis R G. Mechanical properties of acellular peripheral nerve. J Surg Res. 2003 October; 114(2):133-9.
24. Falanga, V. J., Tissue engineering in wound repair. Adv Skin Wound Care, 2000. 13(2 Suppl): p. 15-9.
25. Sung, H. W., W. H. Chang, C. Y. Ma, and M. H. Lee, Crosslinking of biological tissues using genipin and/or carbodiimide. J Biomed Mater Res, 2003. 64A(3): p. 427-38.
26. Liang, H. C., W. H. Chang, K. J. Lin, and H. W. Sung, Genipin-crosslinked gelatin microspheres as a drug carrier for intramuscular administration: in vitro and in vivo studies. J Biomed Mater Res, 2003. 65A(2): p. 271-82.
27. Khor, E., Methods for the treatment of collagenous tissues for bioprostheses. Biomaterials, 1997. 18(2): p. 95-105.
28. Lee, C. R., A. J. Grodzinsky, and M. Spector, The effects of cross-linking of collagen-glycosaminoglycan scaffolds on compressive stiffness, chondrocyte-mediated contraction, proliferation and biosynthesis. Biomaterials, 2001. 22(23): p. 3145-54.
29. Girton, T. S., T. R. Oegema, E. D. Grassl, B. C. Isenberg, and R. T. Tranquillo, Mechanisms of stiffening and strengthening in media-equivalents fabricated using glycation. J Biomech Eng, 2000. 122(3): p. 216-23.
30. Myles, J. L., B. T. Burgess, and R. B. Dickinson, Modification of the adhesive properties of collagen by covalent grafting with RGD peptides. J Biomater Sci Polym Ed, 2000. 11(1): p. 69-86.
31. Enever, P. A., D. I. Shreiber, and R. T. Tranquillo, A novel implantable collagen gel assay for fibroblast traction and proliferation during wound healing. J Surg Res, 2002. 105(2): p. 160-72.
32. Bromberek, B. A., P. A. Enever, D. I. Shreiber, M. D. Caldwell, and R. T. Tranquillo, Macrophages influence a competition of contact guidance and chemotaxis for fibroblast alignment in a fibrin gel coculture assay. Exp Cell Res, 2002. 275(2): p. 230-42.
33. Shreiber, D. I., P. A. J. Enever, and R. T. Tranquillo, Effects of PDGF-BB on rat dermal fibroblast behavior in mechanically stressed and unstressed collagen and fibrin gels. Exp Cell Res, 2001. 266: p. 155-166.
34. Kruse J, Mailhammer R, Wernecke H, Faissner A, Sommer I, Goridis C, Schachner M. Neural cell adhesion molecules and myelin-associated glycoprotein share a common carbohydrate moiety recognized by monoclonal antibodies L2 and FINK-1. Nature. 1984 Sep. 13-19; 311(5982):153-5.
35. Martini R, Xin Y, Schmitz B, Schachner M. The L2/HNK-1 Carbohydrate Epitope is Involved in the Preferential Outgrowth of Motor Neurons on Ventral Roots and Motor Nerves. Eur J Neurosci. 1992; 4(7):628-639.
36. Martini R, Schachner M, Brushart T M. The L2/HNK-1 carbohydrate is preferentially expressed by previously motor axon-associated Schwann cells in reinnervated peripheral nerves. J Neurosci. 1994 November; 14(11 Pt 2):7180-91.
37. Eberhardt K A, Irintchev A, Al-Majed A A, Simova O, Brushart T M, Gordon T, Schachner M. BDNF/TrkB signaling regulates HNK-1 carbohydrate expression in regenerating motor nerves and promotes functional recovery after peripheral nerve repair. Exp Neurol. 2006 April; 198(2):500-10. Epub 2006 Feb. 7.
38. Simova O, Irintchev A, Mehanna A, Liu J, Dihne M, Bachle D, Sewald N, Loers G, Schachner M. Carbohydrate mimics promote functional recovery after peripheral nerve repair. Ann Neurol. 2006 October; 60(4):430-7.
39. Simon-Haldi M, Mantei N, Franke J, Voshol H, Schachner M. Identification of a peptide mimic of the L2/HNK-1 carbohydrate epitope. J Neurochem. 2002 December; 83(6):1380-8.
40. Torregrossa P, Buhl L, Bancila M, Durbec P, Schafer C, Schachner M, Rougon G. Selection of poly-alpha 2,8-sialic acid mimotopes from a random phage peptide library and analysis of their bioactivity. J Biol Chem. 2004 Jul. 16; 279(29):30707-14. Epub 2004 May 6.
41. Bachle D, Loers G, Guthohrlein E W, Schachner M, Sewald N. Glycomimetic cyclic peptides stimulate neurite outgrowth. Angew Chem Int Ed Engl. 2006 Oct. 6; 45(39):6582-5.
42. Chen J., Irintchev A., Rougon G. and Schachner M. (2006) Glycomimetics enhance locomotor recovery after spinal cord injury in adult mice. submitted.
43. Kuijpers, A. J., G. H. Engbers, J. Krijgsveld, S. A. Zaat, J. Dankert, and J. Feijen, Cross-linking and characterisation of gelatin matrices for biomedical applications. J Biomater Sci Polym Ed, 2000. 11(3): p. 225-43.
44. Gratzer, P. F. and J. M. Lee, Control of pH alters the type of cross-linking produced by 1-ethyl-3-(3-dimethylaminopropyl)-carbodiimide (EDC) treatment of acellular matrix vascular grafts. J Biomed Mater Res, 2001. 58(2): p. 172-9.
45. Olde Damink, L. H., P. J. Dijkstra, M. J. van Luyn, P. B. van Wachem, P. Nieuwenhuis, and J. Feijen, Cross-linking of dermal sheep collagen using a water-soluble carbodiimide. Biomaterials, 1996. 17(8): p. 765-73.
46. Song, M., H. Sundararaghavan, D. I. Shreiber, and K. E. Uhrich. Modifying cell adhesion to collagen gels for enhanced nerve regeneration. in BMES. 2004. Philadelphia, Pa.
47. Masuko, Kuromi, and Shimada, Isolation and culture of motoneurons from embryonic chicken spinal cords, PNAS 76(7) 3537-3541, 1979.
48. Simova O, Irintchev A, Mehanna A, Liu J, Dihne M, Bachle D, Sewald N, Loers G, Schachner M. Carbohydrate mimics promote functional recovery after peripheral nerve repair. Ann Neurol. 2006 October; 60(4):430-7.
49. Pego, A. P., Vleggeert-Lankamp, C. L., Deenen, M., Lakke, E. A., Grijpma, D. W., Poot, A. A., Marani, E. Feijen, J., Adhesion and growth of human Schwann cells on trimethylene carbonate (co)polymers. J Biomed Mater Res A, 2003. 67(3): p. 876-885.
50. Morrissey, T. K., Kleitman, N. Bunge, R. P., Isolation and functional characterization of Schwann cells derived from adult peripheral nerve. J Neurosci, 1991. 11(8): p. 2433-2442.
51. Aebischer, P., Guenard, V., Valentini, R. F., The morphology of regenerating peripheral nerves is modulated by the surface microgeometry of polymeric guidance channels, Brain Res. 531:211-218, 1990.
52. Belkas, J. S., Munro, C. A., Shoichet, M. S., Johnston, M., Midha, R., Long-term in vivo biomechanical properties and biocompatibility of poly(2-hydroxyethyl methacrylate-co-methyl methacrylate) nerve conduits, Biomaterials. 26:1741-1749, 2005.
53. Rydevik, B. L., Kwan, M. K., Myers, R. R., Brown, R. A., Triggs, K. J., Woo, S. L., Garfin, S. R., An in vitro mechanical and histological study of acute stretching on rabbit tibial nerve, J Orthop Res. 8:694-701, 1990.
54. Weir, N. A., Buchanan, F. J., Orr, J. F., Farrar, D. F., Boyd, A., Processing, annealing and sterilisation of poly-L-lactide, Biomaterials. 25:3939-3949, 2004.
55. Wang, Y., Ameer, G. A., Sheppard, B. J., Langer, R., A tough biodegradable elastomer, Nat. Biotechnol. 20:602-606, 2002.
56. Thompson D M, Buettner H M. Oriented Schwann cell monolayers for directed neurite outgrowth. Ann Biomed Eng. 2004 August; 32(8):1120-30.

57. Thompson D M, Buettner H M. Schwann cell response to micropatterned laminin surfaces. Tissue Eng. 2001 June; 7(3):247-65.
58. T. Anastasiou and K. Uhrich. Novel polyanhydrides with enhanced thermal and solubility properties. Macromolecules 33:6217-6221 (2000).

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A cell guidance tube that comprises an inner layer, wherein the inner layer comprises at least one biodegradable polymer, wherein the tube comprises a lumen that comprises at least one immobilized peptide mimic of a growth factor.

2. The tube of claim 1 that further comprises an outer layer, wherein the inner layer and the outer layer each comprise at least one biodegradable polymer.

3. A cell guidance tube that comprises an inner layer and an outer layer, wherein the inner layer and the outer layer each comprise at least one biodegradable polymer.

4. The tube of claim 3, wherein the tube comprises a lumen that comprises a peptide mimic of a growth factor.

5. The tube of claim 1, wherein the growth factor is a nerve growth factor.

6. The tube of claim 1, wherein the growth factor is human natural killer cell epitope (HNK-1) or polysialic acid (PSA).

7. The tube of claim 2 that further comprises a middle layer disposed between the inner layer and the outer layer.

8. The tube of claim 7, wherein the middle layer comprises at least one biodegradable polymer.

9. The tube of claim 2, wherein the inner and outer layers have different degradation rates.

10. The tube of claim 1, wherein the inner diameter of the tube is about 2-3 mm.

11. The tube of claim 1, wherein the length of the tube is about 10-30 mm.

12. The tube of claim 1, wherein the tube is formed by extrusion.

13. The tube of claim 1, wherein at least one biodegradable polymer is a bioactive polymer.

14. The tube of claim 1, wherein at least one of the inner, middle, or outer layers comprises a polymer having one or more anti-inflammatory compounds in the polymer backbone.

15. The tube of claim 14, wherein the polymer is a polyanhydride.

16. The tube of claim 14, wherein the polymer is a polyester or a polyamide.

17. The tube of claim 1, wherein at least one of the inner, middle, or outer layers comprises a salicylic acid-based polymer, a salicylsalicylic acid-based polymer, or a difluorophenyl-salicylic acid-based polymer.

18. The tube of claim 1, wherein at least one biodegradable polymer degrades to release an anti-inflammatory compound.

19. The tube of claim 1, wherein at least one biodegradable polymer degrades to release salicylic acid.

20. The tube of claim 1, wherein at least one biodegradable polymer degrades to release a non-steroidal anti-inflammatory compound.

21. The tube of claim 1, wherein at least one of the inner, middle, or outer layers comprises a polymer having one or more antibiotic compounds in the polymer backbone.

22. The tube of claim 21, wherein the polymer is a polyanhydride.

23. The tube of claim 21, wherein the polymer is a polyester or a polyamide.

24. The tube of claim 1, wherein at least one biodegradable polymer degrades to release an antibiotic compound.

25. The tube of claim 1, wherein at least one of the inner, middle, or outer layers comprises a polymer having one or more growth factor compounds in the polymer backbone.

26. The tube of claim 25, wherein the polymer is a polyanhydride.

27. The tube of claim 25, wherein the polymer is a polyester or a polyamide.

28. The tube of claim 1, wherein at least one biodegradable polymer degrades to release a growth factor.

29. The tube of claim 1, wherein the lumen comprises collagen, chitosan, agarose or gelatin.

30. The tube of claim 29, wherein the lumen comprises collagen.

31. The tube of claim 1, wherein the lumen comprises longitudinal channels.

32. The tube of claim 1, wherein at least one of the inner, middle, or outer layers comprises a xylyl-based polymer.

33. The tube of claim 1, wherein at least one of the inner, middle, or outer layers comprises an iodinated salicylate-based polymer.

34. A cell guidance tube that comprises an inner layer, wherein the inner layer comprises at least one biodegradable polymer, wherein the tube comprises a lumen that comprises at least one immobilized peptide mimic of a carbohydrate.

35. A method for regenerating a damaged nerve in a patient in need thereof, comprising placing the cell guidance tube of claim 1 at a site of neuronal injury so as to regenerate the nerve.

36. The method of claim 35, wherein the nerve is a peripheral nerve.

37. (canceled)

38. (canceled)
